(19) United States
(12) Patent Application Publication
(10) Pub. No.: US 2025/0148600 A1
(43) Pub. Date: May 8, 2025

(54) BED INDUCTION SYSTEM, MEDICAL APPARATUS, BED, AND MEDICAL IMAGE DIAGNOSIS SYSTEM

(71) Applicant: FUJIFILM Corporation, Tokyo (JP)

(72) Inventor: Satoshi IIZUKA, Tokyo (JP)

(73) Assignee: FUJIFILM Corporation, Tokyo (JP)

(21) Appl. No.: 18/939,498

(22) Filed: Nov. 6, 2024

(30) Foreign Application Priority Data
Nov. 7, 2023 (JP) 2023-190229

(51) Int. Cl. G06T 7/00; A61B 5/00; G16H 30/40

(52) U.S. Cl. CPC G06T 7/0012; A61B 5/0033; A61B 5/7425; G16H 30/40

(57) ABSTRACT

Provided are a bed induction system, a medical apparatus, a bed, and a medical image diagnosis system that can reduce an operation load of a dockable bed. A bed induction system includes one or more sensors that obtain information on a positional relationship between a medical apparatus having a docking portion to which a bed is connected and the bed, one or more processors that execute processing of displaying guide information for inducing the bed to the docking portion based on the information obtained from the one or more sensors, and one or more display devices that display the guide information.

### CROSS-REFERENCE TO RELATED APPLICATION

The present application claims priority under 35 U.S.C § 119(a) to Japanese Patent Application No. 2023-190229 filed on Nov. 7, 2023, which is hereby expressly incorporated by reference, in its entirety, into the present application.

### BACKGROUND OF THE INVENTION

### 1. Field of the Invention

The present disclosure relates to a bed induction system, a medical apparatus, a bed, and a medical image diagnosis system and particularly to a technique of supporting control of the bed in a case of docking a mobile bed, which is attachable to and detachable from the medical apparatus, to the medical apparatus.

### 2. Description of the Related Art

A medical image diagnosis system, such as a magnetic resonance imaging (MRI) apparatus and a computed tomography (CT) apparatus, has a structure in which a subject placed on a bed is inserted into an imaging space of a medical imaging apparatus main body called a gantry to perform imaging. US2022/0047218A, US2016/0066869A, and JP6002676B disclose a mobile bed that is attachable to and detachable from an apparatus body of the MRI apparatus.

### SUMMARY OF THE INVENTION

A docking portion, which is a connection mechanism with respect to the bed, is provided at the gantry to which the movable dockable bed is connected, and the connection mechanism that engages with a docking portion on a gantry side is provided at the bed. In a case where an operator of the bed moves the bed to a docking position, it is necessary to control the bed while being careful not to accidentally hit the bed against a cover of the gantry or the like. In general, the docking portion is disposed at a low position near a lower portion of a gantry front surface, and in a case where the bed approaches the gantry, the docking portion is hardly visible from the operator who pushes the bed. In a medical site, the operator of the bed tends to confirm a position while visually checking the docking portion on the gantry side and to carefully control the bed.

On the other hand, the connecting mechanism adopted in the docking portion on the gantry side has a guide mechanism that enables docking insofar as an angle at which the bed enters is within a certain angle range (see JP6002676B). For this reason, the bed does not need to face the docking portion of the gantry from the front and is mechanically guided and connected by the docking portion insofar as the bed roughly travels within a certain allowable angle range.

However, even in a case of comprising the connection mechanism having an allowable angle range for an approach angle of such a bed, the bed is carefully controlled from the viewpoint of a general operator.

The present disclosure has been devised in view of such circumstances, and an object thereof is to provide a bed induction system, a medical apparatus, a bed, and a medical image diagnosis system that can reduce an operation load of a dockable bed.

According to a first aspect of the present disclosure, there is provided a bed induction system comprising one or more sensors that obtain information on a positional relationship between a medical apparatus having a docking portion to which a bed is connected and the bed, one or more processors that execute processing of displaying guide information for inducing the bed to the docking portion based on the information obtained from the one or more sensors, and one or more display devices that display the guide information.

According to the first aspect, the positional relationship between the medical apparatus and the bed is ascertained using the one or more sensors, and the guide information for inducing the bed to the docking portion is displayed on the one or more display devices based on the information obtained from the one or more sensors. An operator of the bed can dock the bed to the medical apparatus by controlling the bed in accordance with the guide information. An operation load in a case of docking control is reduced by the display of the guide information.

According to a second aspect, in the bed induction system according to the first aspect, the medical apparatus may be a gantry of a medical imaging apparatus that captures a medical image of a subject.

According to a third aspect, in the bed induction system according to the first or second aspect, the one or more sensors may be configured to be disposed at at least one of the medical apparatus or the bed.

According to a fourth aspect, in the bed induction system according to any one of the first to third aspects, the one or more sensors may be configured to be disposed at at least one of a ceiling or a wall of a room where the medical apparatus is disposed.

According to a fifth aspect, in the bed induction system according to any one of the first to fourth aspects, the one or more sensors may be configured to include a camera.

According to a sixth aspect, in the bed induction system according to the fifth aspect, the one or more processors may be configured to ascertain the positional relationship between the medical apparatus and the bed by analyzing an image obtained from the camera.

According to a seventh aspect, in the bed induction system according to any one of the first to sixth aspects, the one or more display devices may be configured to be disposed at at least one of the medical apparatus or the bed.

According to an eighth aspect, in the bed induction system according to any one of the first to the seventh aspects, the one or more processors may be configured to generate the guide information based on the information obtained from the one or more sensors.

According to a ninth aspect, in the bed induction system according to any one of the first to eighth aspects, the guide information may be configured to include information indicating a traveling route for guiding the bed to the docking portion.

According to a tenth aspect, in the bed induction system according to any one of the first to ninth aspects, the guide information may be configured to include information indicating a dockable angle range of the bed with respect to the docking portion.

According to an eleventh aspect, in the bed induction system according to any one of the first to tenth aspects, the information on the positional relationship may be configured to include distance information between the medical apparatus and the bed.

According to a twelfth aspect, in the bed induction system according to any one of the first to eleventh aspects, the one or more processors may be configured to display the guide information on the one or more display devices in a case where a distance between the medical apparatus and the bed is a first distance or less.

According to a thirteenth aspect, in the bed induction system according to the twelfth aspect, the one or more processors may be configured to, in a case where display of the guide information on the one or more display devices is started, maintain the display of the guide information until docking completion.

According to a fourteenth aspect, in the bed induction system according to any one of the first to thirteenth aspects, the one or more processors may be configured to hide the guide information in a case of undocking, which is separating the bed from the medical apparatus, and activate a guide display function after a predetermined time elapses from the undocking.

According to a fifteenth aspect, in the bed induction system according to any one of the first to fourteenth aspects, the bed may be configured to include an electrically assisted caster that provides traveling assistance to the bed, and the one or more processors may be configured to perform control of reducing the traveling assistance of the electrically assisted caster in a case where a distance between the medical apparatus and the bed is a second distance or less.

Stopping an assistance function by the electrically assisted caster includes a concept of reducing the traveling assistance.

According to a sixteenth aspect, in the bed induction system according to any one of the first to fifteenth aspects, the bed may be configured to include a steering caster that changes a traveling direction of the bed, and the one or more processors may be configured to control the steering caster so that the bed is directed to the docking portion based on the information obtained from the one or more sensors.

According to a seventeenth aspect, there is provided a medical apparatus comprising a docking portion to which a bed is connected, one or more sensors that obtain information on a positional relationship with the bed, one or more processors that execute processing of displaying guide information for inducing the bed to the docking portion based on the information obtained from the one or more sensors, and one or more display devices that display the guide information.

In the medical apparatus according to the seventeenth aspect, a configuration where the same specific aspects as that of the bed induction system according to the second to sixteenth aspects can be adopted.

According to an eighteenth aspect, there is provided a bed comprising one or more sensors that obtain information on a positional relationship between the medical apparatus and the bed, one or more processors that execute processing of displaying guide information for inducing the bed to the docking portion based on the information obtained from the one or more sensors, and one or more display devices that display the guide information.

In the bed according to the eighteenth aspect, a configuration where the same specific aspects as that of the bed induction system according to the second to sixteenth aspects can be adopted.

According to a nineteenth aspect, there is provided a medical image diagnosis system comprising a gantry that has a docking portion to which a bed is connected, the bed that is attachable to and detachable from the docking portion, one or more sensors that obtain information on a positional relationship between the gantry and the bed, one or more processors that execute processing of displaying guide information for inducing the bed to the docking portion based on the information obtained from the one or more sensors, and one or more display devices that display the guide information.

In the medical image diagnosis system according to the nineteenth aspect, a configuration where the same specific aspects as that of the bed induction system according to the second to sixteenth aspects can be adopted.

With the present disclosure, an operation load of the bed that is attachable to and detachable from the medical apparatus can be reduced.

### BRIEF DESCRIPTION OF THE DRAWINGS

FIG. 1 is a perspective view showing a configuration example of a medical image diagnosis system to which a bed induction system according to a first embodiment is applied.

FIG. 2 is a block diagram schematically showing a configuration of the bed induction system according to the first embodiment.

FIG. 3 is an explanatory view showing an example of an angle range of an allowable bed approach angle with respect to a docking portion of a gantry and an example of a distance that is a determination criterion as to whether or not to start display of guide information.

FIG. 4 is an explanatory view showing a display example of the guide information.

FIG. 5 is a flowchart showing an example of an operation of the bed induction system according to the first embodiment.

FIG. 6 is a perspective view showing a configuration of a medical image diagnosis system according to modification example 1 of the first embodiment.

FIG. 7 is a perspective view of a bed used in a second embodiment.

FIG. 8 is a functional block diagram of a medical image diagnosis system according to the second embodiment.

FIG. 9 is a schematic side view schematically showing a configuration of a bed according to a third embodiment.

FIG. 10 is a functional block diagram of a bed induction system mounted on the bed according to a third embodiment.

FIG. 11 is a flowchart showing an example of an operation of the bed induction system according to the third embodiment.

FIG. 12 is a perspective view showing a structural example of a bed comprising an electrically assisted caster and a steering caster.

FIG. 13 is a functional block diagram of the bed comprising the electrically assisted caster and the steering caster.

FIG. 14 is a flowchart showing example 1 of an operation of the bed comprising the electrically assisted caster and the steering caster.

FIG. 15 is a flowchart showing example 2 of the operation of the bed comprising the electrically assisted caster and the steering caster.

### DESCRIPTION OF THE PREFERRED EMBODIMENTS

Hereinafter, preferred embodiments of the present invention will be described in detail with reference to the accompanying drawings.

### First Embodiment

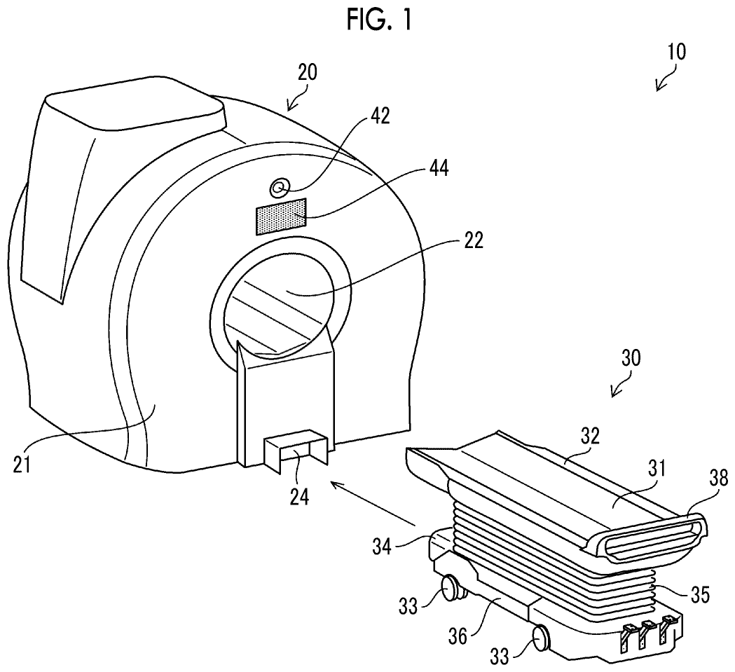

FIG. 1 is a perspective view showing a configuration example of a medical image diagnosis system 10 to which a bed induction system according to a first embodiment is applied. The medical image diagnosis system 10 includes a gantry 20 that is a medical imaging apparatus main body which captures a medical image of a subject and a mobile bed 30 that is attachable to and detachable from the gantry 20.

The gantry 20 may have any configuration insofar as the gantry 20 is configured to capture an image of the subject mounted on the bed 30. Herein, a case where the medical image diagnosis system 10 is an MRI apparatus will be described as an example, but without being limited to the MRI apparatus, the medical image diagnosis system 10 may be, for example, a CT apparatus, a positron emission tomography (PET) apparatus, or other modality apparatus. The gantry 20 is an example of a “medical apparatus” according to the embodiment of the present disclosure.

Although a detailed configuration of the gantry 20 of the MRI apparatus is not shown, the gantry 20 is composed of a static magnetic field generation device that generates a static magnetic field, a gradient magnetic field coil, a high radio frequency (RF) magnetic field pulse transmission coil, a shim coil for static magnetic field correction, and a gantry cover 21 that covers these elements. The static magnetic field generation device of the gantry 20 shown in FIG. 1 has a cylindrical shape of which an axial direction is horizontal, and an inner space of the cylinder is an imaging space 22. However, the present invention is not limited to the cylindrical static magnetic field generation device.

The MRI apparatus further comprises, as a power source, a control and signal processing system (not shown), a gradient magnetic field power amplifier that supplies a current to the gradient magnetic field coil, a high frequency power amplifier that supplies a high frequency signal to the RF magnetic field pulse transmission coil, a high frequency amplification circuit, a computer, an operating part, and a display.

The gantry 20 is disposed in an electromagnetic shielded room (for example, an examination room). The power source and the control and signal processing system are disposed outside the electromagnetic shielded room and are electrically connected to the gantry 20 by a cable.

The static magnetic field generation device generates a static magnetic field in the imaging space 22, and the shim coil generates a magnetic field for improving homogeneity of the static magnetic field to a predetermined value or more. The gradient magnetic field coil generates a gradient magnetic field in each of predetermined XYZ-directions in the imaging space 22. The RF magnetic field pulse transmission coil transmits an RF magnetic field pulse to the imaging space 22.

The computer outputs control signals to the gradient magnetic field power amplifier, the high frequency power amplifier, and the high frequency amplification circuit and controls an application timing and a direction of the gradient magnetic field, an irradiation timing of the RF magnetic field pulse, and the like in accordance with a predetermined imaging sequence. Accordingly, a nuclear magnetic resonance (NMR) signal generated from the subject is received by a reception coil disposed near the subject. The high frequency amplification circuit detects and amplifies the signal under the control of the computer. The computer performs processing such as reconstructing an image from the obtained signal in accordance with a predetermined image reconstruction program and displaying the image on the display. The operating part receives an imaging condition and the like from an examiner.

The bed 30 comprises a top plate 31 for mounting the subject, a top plate holding part 32 that holds the top plate 31, a frame that holds the top plate holding part 32 to be movable up and down, an up and down driving unit that moves the top plate holding part 32 up and down, a horizontal drive mechanism for moving the top plate 31 in a horizontal direction with respect to the top plate holding part 32, a horizontal driving unit that drives the horizontal drive mechanism, a wheel 33 that is attached to a lower portion of the frame, a bellows portion 35 and a cover 36 that cover an outer periphery of the frame, and a handle portion 38. With these configurations, the bed 30 can be inserted into an imaging region by raising the top plate 31 to the height of the imaging space 22 of the gantry 20 and sliding the top plate 31 in the horizontal direction with respect to the top plate holding part 32. Accordingly, an imaging site of the subject is transported to the center of the imaging space 22.

The bed 30 is a dockable bed that can be separated and moved from the gantry 20, and an operator of the bed 30 can move the bed 30 by pushing the handle portion 38 of the bed 30 by hand. For example, the bed 30 can be moved to a front room, a patient's room of the subject, or the like that is not affected by magnetism of the gantry 20, and the subject can be mounted on the bed 30. After mounting the subject, the bed 30 is moved in a state where the subject is mounted and can be docked to the gantry 20.

A docking portion 24 that is a connection mechanism which mechanically and electrically connects the bed 30 is provided on a front surface of the gantry 20. A docking portion 34 that is a connection mechanism which is attachable to and detachable from the docking portion 24 of the gantry 20 is provided on a front side edge portion of the bed 30. The docking portion 34 of the bed 30 is fitted to the docking portion 24 of the gantry 20, thereby the bed 30 is connected (docked) to the gantry 20. As for mechanical structures of the docking portion 24 and the docking portion 34, for example, structures described in JP6002676B or the like can be applied. The docking portion 24 comprises a guide mechanism having a mechanical structure that guides the movement of the bed 30 to enable docking in a case where an approach angle of the bed 30 is within a certain angle range.

The medical image diagnosis system 10 comprises a camera 42 and a display device 44 as a bed induction system that supports control of the bed 30 in a case of docking the bed 30 to the gantry 20 and displays guide information for inducing the bed 30 to the docking portion 24 of the gantry 20 on the display device 44 based on a positional relationship between the bed 30 and the gantry 20 ascertained from an image captured by the camera 42. The term “bed induction system” includes, for example, a concept of a term, such as a docking assistance system, a bed control support system, and a docking induction system, and a system name may be replaced with these terms.

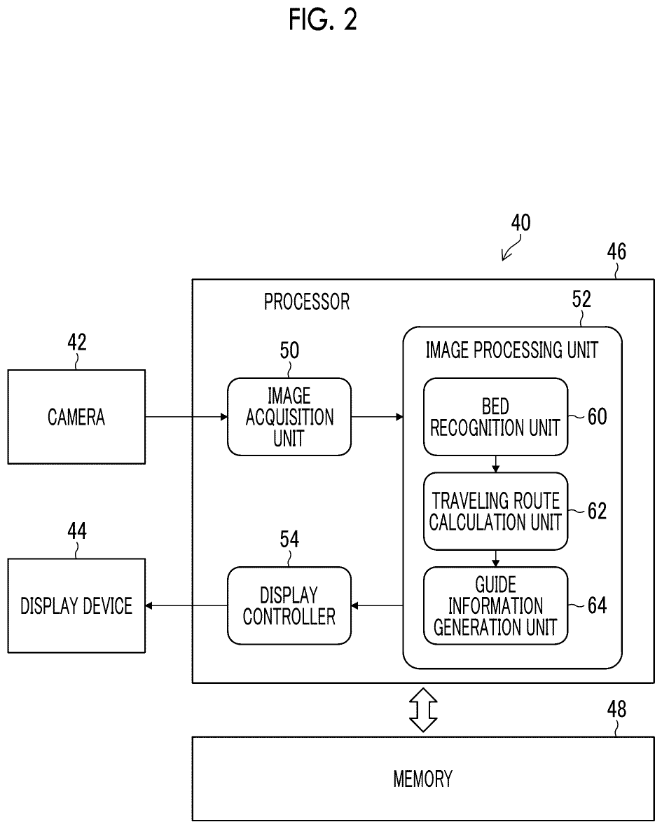

FIG. 2 is a block diagram schematically showing a configuration of a bed induction system 40 incorporated in the gantry 20. The bed induction system 40 is composed of the camera 42, the display device 44, a processor 46, and a memory 48. The camera 42 is an example of a sensor from which information on a positional relationship between the gantry 20 and the bed 30 is obtained.

The camera 42 typically includes an imaging optical system including one or more lenses and an imaging element that captures an optical image formed by the imaging optical system and that converts the optical image into an electric signal. The imaging element is composed of, for example, a complementary metal-oxide semiconductor (CMOS) type color image sensor. The imaging element is not limited to a CMOS type image sensor and may be an XY address type image sensor or a charge coupled device (CCD) type image sensor. In addition, the camera 42 may be composed of an image processing circuit that processes the electric signal obtained from the imaging element to form a digital image.

The camera 42 is attached to, for example, an upper portion of the front surface of the gantry 20 (see FIG. 1). It is preferable that the camera 42 is disposed near directly above the docking portion 24 so that the camera 42 can image the bed 30 approaching the docking portion 24 of the gantry 20 from the front if possible. There may be one or a plurality of cameras 42. By using the plurality of cameras, it is possible to more accurately ascertain the position of the bed 30.

The display device 44 may be, for example, a gantry monitor attached to the front surface of the gantry 20. A screen size of the display device 44 is preferably a large screen size so that the operator who pushes the handle portion 38 of the bed 30 can easily visually recognize display content of the display device 44 from the rear of the bed 30. The number of display devices 44 is not limited to one and there may be a plurality of display devices 44.

The processor 46 includes a central processing unit (CPU). The processor 46 may be composed of one or more processors, such as a graphics processing unit (GPU), a digital signal processor (DSP), an application specific integrated circuit (ASIC), and a programmable logic device (PLD).

The memory 48 includes a random access memory (RAM). The memory 48 may include a read only memory (ROM). The memory 48 stores a program, data, and the like for the processor 46 to realize various types of functions. The processor 46 functions as various types of processing sections by executing instructions stored in the memory 48. The bed induction system 40 may include a storage (not shown). The storage may be, for example, a hard disk drive (HDD), a solid state drive (SSD), or a combination of a plurality thereof. In addition, the storage may include an external storage device, such as a removable medium.

The processor 46 and the memory 48 may be mounted on a control substrate in the gantry 20. In addition, a part or all of the processor 46 or the memory 48 may be configured to be included in a computer connected to the gantry 20.

The processor 46 performs processing of recognizing the bed 30 from an image captured by the camera 42, ascertaining a positional relationship between the gantry 20 and the bed 30 through image analysis, and displaying guide information, such as a traveling route for inducing the bed 30 to the docking portion 24, on the display device 44.

The bed induction system 40 may further comprise a sound output device (not shown), and the processor 46 may perform processing of outputting a notification sound from the sound output device in a case where the bed 30 deviates from a traveling route and may notify the operator of the bed 30 with sound.

In addition, once the angle of the bed 30 is within a dockable angle range, the processor 46 may provide a notification with the display device 44 and/or the sound output device.

The processor 46 functions as an image acquisition unit 50, an image processing unit 52, and a display controller 54. The image acquisition unit 50 acquires an image captured by the camera 42. The image processing unit 52 processes the image obtained from the camera 42 (hereinafter, referred to as a “camera image”). The image processing unit 52 includes a bed recognition unit 60, a traveling route calculation unit 62, and a guide information generation unit 64.

The bed recognition unit 60 recognizes the bed 30 from a camera image and ascertains a positional relationship between the gantry 20 and the bed 30. The bed recognition unit 60 can ascertain a position and a posture of the bed 30 captured in the camera image by analyzing the camera image. The bed recognition unit 60 may be configured to include, for example, a trained model trained to perform thing detection using a machine learning algorithm or may be configured to perform image recognition processing such as pattern matching.

Since the position of the camera 42 attached to the gantry 20 is fixed and a positional relationship between the camera 42 and the docking portion 24 is unchanged (fixed), the bed recognition unit 60 can ascertain a positional relationship between the docking portion 24 of the gantry 20 and the docking portion 34 of the bed 30. The ascertaining of a positional relationship between the gantry 20 and the bed 30 includes a concept of ascertaining of the positional relationship between the docking portion 24 and the docking portion 34. The bed recognition unit 60 acquires information, such as a relative position of the bed 30 with respect to the gantry 20 and a posture and a traveling direction of the bed 30.

A method of measuring a distance to a subject in an image through image analysis may be, for example, a configuration where a predetermined position captured by the camera 42 is used as a specific distance. Data of a relationship between the predetermined position and the specific distance may be acquired by performing calibration in a case of system introduction.

The traveling route calculation unit 62 calculates a desired traveling route as a route for moving the bed 30 to the docking position with the docking portion 24 from a positional relationship between the gantry 20 and the bed 30 ascertained by the bed recognition unit 60. In the calculation of the traveling route, an angle range of an approach angle allowed by the docking portion 24 is considered.

The guide information generation unit 64 generates guide information for inducing the bed 30 to the docking portion 24 of the gantry 20 based on calculation results of the traveling route calculation unit 62. The guide information may be, for example, an arrow or a line indicating a desired (ideal) traveling route of the bed 30, an arrow indicating a desired traveling direction of the bed 30, a line indicating a range of an allowable traveling direction, or an appropriate combination thereof. The guide information is not limited to a figure such as an arrow and a line and may include a character, a symbol, and a mark. The guide information is not limited to information that is adaptively (dynamically) generated from a positional relationship between the gantry 20 and the bed 30 and may include, for example, fixed information, such as a line indicating a dockable angle range.

The display controller 54 generates a display signal necessary for display output to the display device 44 and controls display of the display device 44. The display controller 54 displays guide information on the display device 44 based on processing results of the image processing unit 52. Display of the guide information, the bed 30, and the like on the display device 44 may be performed, for example, as an illustration, may be superimposed on a navigation line or the like in a live-action video (camera image) taken by the camera 42, or may be a combination thereof.

It is preferable that guide information for supporting such docking control is displayed in a case where the bed 30 has approached the gantry 20 within a predetermined distance. The processor 46 recognizes that the bed 30 has approached the gantry 20, starts processing of guide display, and displays the guide information on the display device 44.

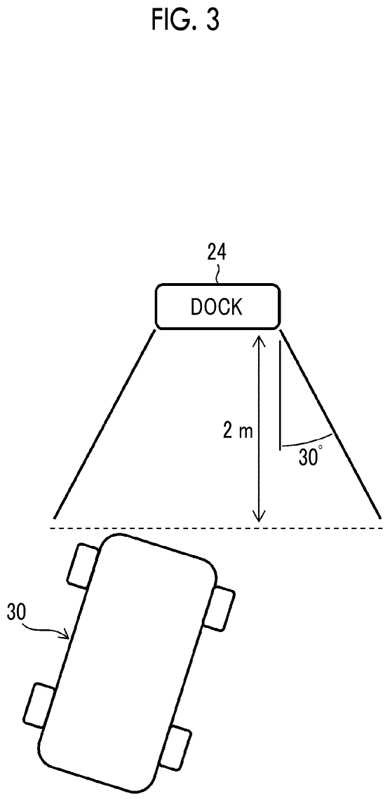

FIG. 3 is an explanatory view showing an example of an angle range of an allowable bed approach angle with respect to the docking portion 24 of the gantry 20 and an example of a predetermined distance that determines a start timing of guide display. A dockable angle range of the docking portion 24 can be determined in advance as a fixed value. As shown in FIG. 3, the approach angle of the bed 30 with respect to the docking portion 24 is allowed in an angle range of 30 degrees on, for example, each of a right side and a left side with the front (0 degrees) as reference, and the bed 30 that enters at an approach angle in the angle range can be docked to the docking portion 24. Such an angle range is determined by the mechanical structure of the docking portion 24. In addition, this angle range may be determined in consideration of a position and a size of the bed 30, a size of a room where the gantry 20 is placed, a position of an entrance and an exit, and the like.

A distance between the gantry 20 and the bed 30, which is a criterion for determining whether or not to start display of guide information, can also be set to an appropriate value (predetermined distance) in advance. For example, the predetermined distance may be set to 2 m. 2 m, which is the predetermined distance, is an example of a “first distance” according to the embodiment of the present disclosure. In this case, in a case where the distance between the gantry 20 and the bed 30 has approached 2 m or less, the display of the guide information is started.

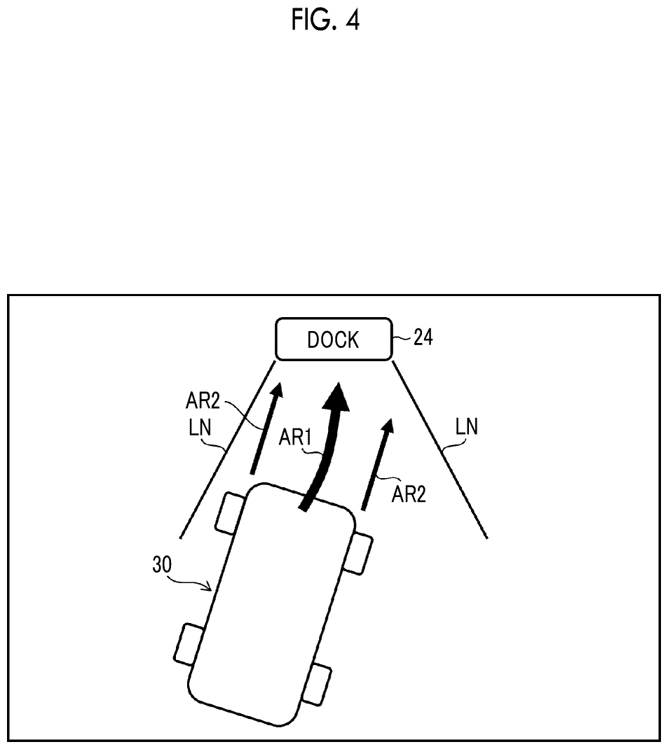

FIG. 4 is a view showing a display example of guide information. In the display device 44, for example, as shown in FIG. 4, the guide information for inducing the bed 30 to the docking portion 24 is displayed. The processor 46 recognizes that the bed 30 has approached the gantry 20 within a predetermined distance, performs image analysis for inducing the bed 30 to the docking portion 24, and displays an arrow AR1 indicating an ideal traveling route of the bed 30 with respect to a dockable angle range. An illustration or a motion picture, which is a live-action video, of the bed 30 showing a positional relationship between the docking portion 24 and the bed 30 is displayed on the display device 44 in accordance with image analysis results.

For example, the bed 30 is shown in a real-time motion picture in a live-action video, and the arrow AR1 and/or a line indicating an ideal traveling route is displayed to be superimposed on the live-action video. In addition to the arrow AR1 or instead of the arrow AR1, an arrow AR2 indicating the traveling direction of the bed 30 may be displayed.

In addition, in FIG. 4, an example is shown in which a line LN indicating a dockable angle range with respect to the docking portion 24 is displayed together with the arrows AR1 and AR2. Although a form in which the line LN indicating the dockable angle range is not displayed in guide display is also possible, it is desirable to display the line LN indicating the dockable angle range with respect to the docking portion 24 in order to notify the operator that there is no problem even in a case where the bed 30 enters the docking portion 24 obliquely.

A display form of information indicating a dockable angle range is not limited to display by the line LN and may be, for example, a figure or a coloring indicating an angle range. The information indicating the dockable angle range may be displayed together with display of the arrow AR1 and/or the arrow AR2 or the like, or guide information indicating the angle range may be displayed without displaying the arrow AR1 and/or the arrow AR2.

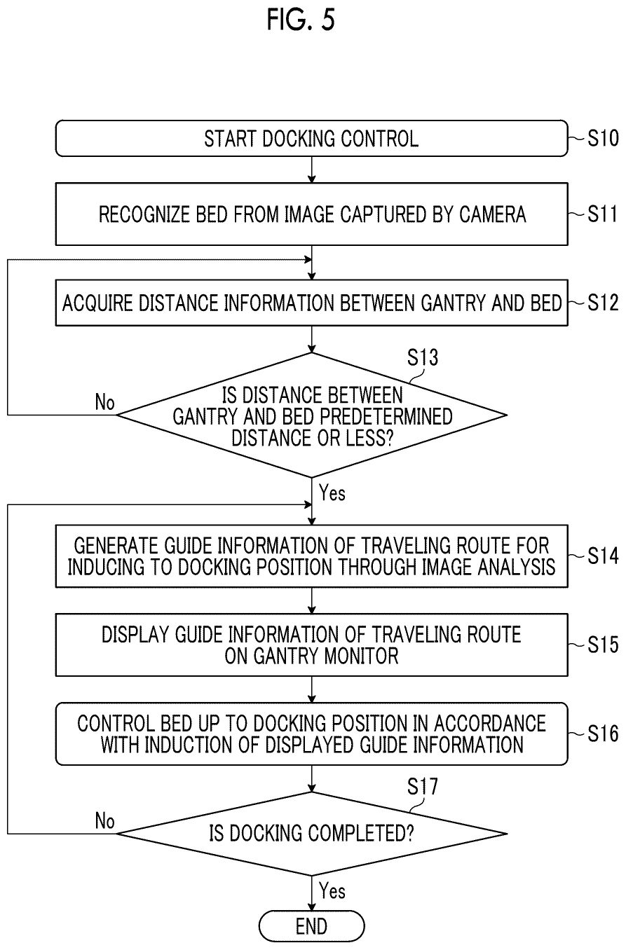

FIG. 5 is a flowchart showing an example of an operation of the bed induction system 40 according to the first embodiment. In step S10, the operator of the bed 30 starts control (docking control) of the bed 30 for docking the bed 30 to the gantry 20. The bed 30 may be configured to travel as a force (manual force) is applied by the operator. A configuration where the bed 30 comprises an electrically assisted caster and the bed 30 travels with a combination of a manual force and motive power of the electrically assisted caster.

In step S11, the processor 46 acquires an image captured by the camera 42 and recognizes the bed 30 from the camera image.

In step S12, the processor 46 ascertains a positional relationship between the gantry 20 and the bed 30 from the camera image and acquires distance information between the gantry 20 and the bed 30.

In step S13, the processor 46 determines whether or not a distance between the gantry 20 and the bed 30 has approached a predetermined distance or less. The predetermined distance may be a distance set as a guide display start determination distance described with reference to FIG. 3. In a case where the determination result in step S13 is No, the processor 46 proceeds to step S12.

In a case where the determination result in step S13 is Yes, the processor 46 proceeds to step S14.

In step S14, the processor 46 generates guide information indicating a traveling route for inducing the bed 30 to the docking position through image analysis on the camera image.

In step S15, the processor 46 displays the guide information on the gantry monitor (the display device 44).

In step S16, the operator controls the bed 30 up to the docking position in accordance with induction of the guide information displayed on the gantry monitor. The bed 30 moves toward the docking position with a manual force of the operator or a combination of the manual force and the motive power of the electrically assisted caster.

In step S17, the processor 46 determines whether or not the docking is completed. In a case where the determination result in step S17 is No (docking is not completed), the processor 46 returns to step S14.

In a case where the determination result in step S17 is Yes (docking completion), the processor 46 ends the guide display.

### Difference in Display Control Between Case of Docking and Case of Undocking

An operation of docking the bed 30 to the gantry 20 is performed before a medical image is captured. In a case of controlling the bed 30 for the docking (in a case of docking control), display of the guide information is started once the bed 30 has approached the gantry 20 by a predetermined distance (for example, 2 m). After the guide display is started, the guide display is maintained until docking is completed. That is, the processor 46 detects the movement of the bed 30 from the camera image and, in a case where the bed 30 has approached in a direction of the gantry 20, displays the guide information even in a case where the bed 30 moves backward halfway after reaching the predetermined distance.

In a case where presence or absence of guide display is determined only by forward movement or backward movement of the bed 30, the guide display is turned off in a case where the bed 30 is temporarily moved backward. In a case where the on/off of the guide display is repeated during the control of the bed 30, there is a concern that the screen display is difficult to see and is felt to be complicated.

For this reason, an aspect in which before a medical image is captured, display of guide information is continued until docking is completed once the display of the guide information is started is preferable for the operator.

Then, in a case of an undocking operation of separating the bed 30 from the gantry 20 after capturing of the medical image, since the display of the guide information as in the case of docking is not necessary, a guide display function may be turned off (deactivated). That is, the processor 46 hides the guide information in a case of undocking.

After undocking, in a case where the next medical imaging is performed, a docking operation of the bed 30 is performed again, and in this case, display of guide information is necessary. Therefore, it is preferable that the processor 46 activates the guide display function after a predetermined time elapses after the undocking. In this case, even in a case where the guide display function is activated, the guide information is actually displayed when the bed 30 has approached the gantry 20 by a predetermined distance.

Switching of activating and deactivating of the guide display function may be performed automatically, or an input of an instruction to activate or deactivate the guide display function may be received via a user interface so that the operator can perform the operation.

### Example 1 of Additional Configuration

In a case where it is detected that there is an obstacle between the gantry 20 and the bed 30 from a camera image, the processor 46 may display the fact on the display device 44 or may perform notification with sound.

### Advantages of First Embodiment

According to the first embodiment, a positional relationship between the bed 30 and the gantry 20 is ascertained using the camera 42, and guide information for inducing the bed 30 to the docking position with the docking portion 24 is displayed on the display device 44. In a case where the operator of the bed 30 controls the bed 30 in accordance with the guide information, the bed 30 can be appropriately docked. An operation load in a case of docking control is reduced by the guide display.

In addition, by displaying information on a dockable angle range as guide information, the operator can easily understand that the operator may make the bed 30 enter in an allowable angle range. For this reason, the operator can understand that an adjustment operation of an exact approach angle is not required and can control the bed 30, reducing a load.

### Modification Example 1

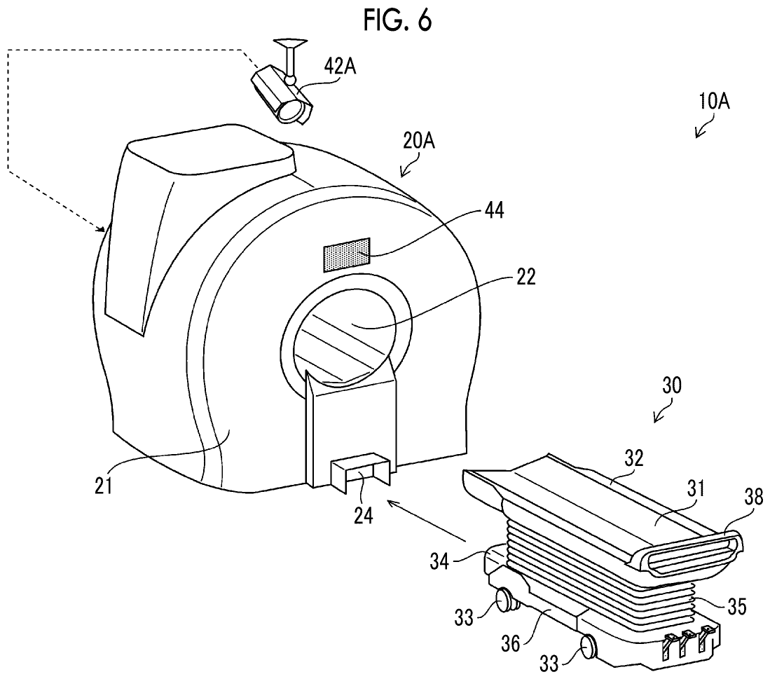

In FIG. 1, although the gantry 20 comprising the camera 42 is given as an example, the camera 42 may be disposed at a location other than the gantry 20. FIG. 6 is a perspective view showing a configuration of a medical image diagnosis system 10A according to modification example 1 of the first embodiment. In the configuration shown in FIG. 6, points different from FIG. 1 will be described.

In the medical image diagnosis system 10A shown in FIG. 6, a camera 42A is disposed on a ceiling of a room where a gantry 20A is disposed, instead of the camera 42. The camera 42A may be disposed at a position where the bed 30 approaching the gantry 20A can be imaged, without being limited to the ceiling, may be attached to a wall of the room, or may be attached to a structure disposed in the room. It is desirable that the camera 42A is disposed near directly above the docking portion 24. Other configurations may be the same as those of the medical image diagnosis system 10 according to the first embodiment.

### Modification Example 2

A sensor other than the camera may be used instead of the camera 42 shown in FIG. 1 or in combination with the camera 42. As a sensor other than the camera, for example, a noncontact distance sensor typified by a triangulation system laser distance sensor can be used.

For example, a configuration where information indicating a distance between the gantry 20 and the bed 30 is acquired by the laser distance sensor, and once the bed 30 has approached the gantry 20 by a predetermined distance or less, guide information including a dockable angle range is displayed may be adopted.

### Second Embodiment

In addition to the configuration of the medical image diagnosis system 10 or the medical image diagnosis system 10A, guide information for supporting docking control may be displayed on a display device on a bed 30 side.

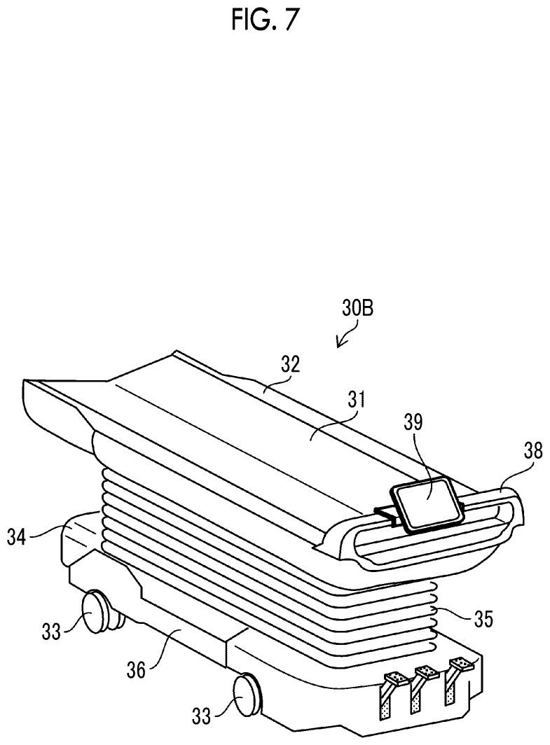

FIG. 7 is a perspective view of a bed 30B used in a second embodiment. Instead of the bed 30 described with reference to FIG. 1, the bed 30B shown in FIG. 7 can be used. The bed 30B comprises an operation panel 39 comprising a display. The operation panel 39 is disposed on, for example, the handle portion 38 of the bed 30B. The operation panel 39 may be configured to be detachable from the handle portion 38 or may be a device such as a tablet terminal. The bed 30B and the gantry 20 are configured to deliver information to each other, for example, through wireless communication, such as Bluetooth (registered trademark).

The bed 30B acquires guide information generated by the processor 46 through wireless communication and displays the guide information on the operation panel 39. Content displayed on the operation panel 39 may be a duplication of the content displayed on the display device 44 (gantry monitor) on a gantry 20 side, may be content processed for display on the operation panel 39, or may include display other than the guide information. As other content displayed on the operation panel 39, a distance to the gantry 20, a battery remaining amount, display indicating “docking completion” in a case of docking completion, and the like may be included.

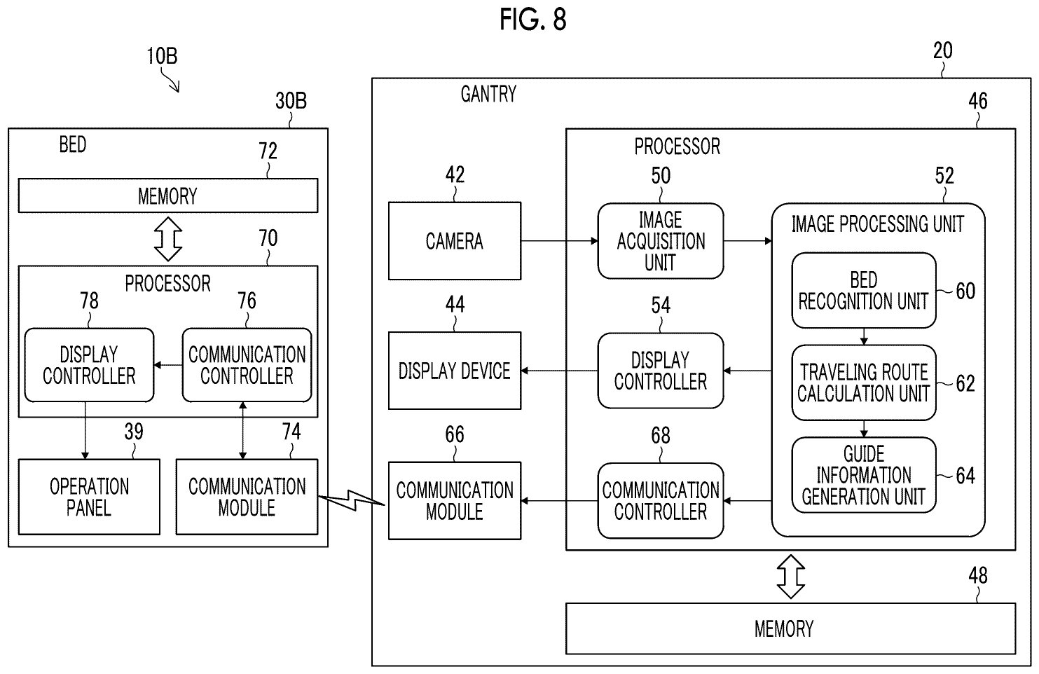

FIG. 8 is a functional block diagram of a medical image diagnosis system 10B according to the second embodiment. Regarding the configuration shown in FIG. 8, elements common to those in FIG. 2 are assigned with the same reference numerals, and redundant description will be omitted. In addition to the configuration described with reference to FIG. 2, the gantry 20 shown in FIG. 8 comprises a communication module 66 for wireless communication, and the processor 46 functions as a communication controller 68. The communication controller 68 controls communication performed by the communication module 66. The communication controller 68 performs control of transmitting information necessary for guide display from the communication module 66 based on processing results of the image processing unit 52.

The bed 30B comprises the operation panel 39, a processor 70, a memory 72, and a communication module 74 for wireless communication. The processor 70 and the memory 72 may have the same configurations as those of the processor 46 and the memory 48. The memory 72 stores a program, data, and the like for causing the processor 70 to realize various types of functions. A part or all of the processor 70 and the memory 72 may be configured to be mounted on a control substrate in the bed 30B.

The processor 70 functions as a communication controller 76 and a display controller 78. The communication controller 76 controls communication performed by the communication module 74. The communication modules 66 and 74 may be composed of a signal processing circuit that performs a part or all of processing functions of the communication controllers 68 and 76, respectively.

The communication module 74 communicates with the communication module 66 in accordance with a predetermined communication protocol and acquires information necessary for guide display from the gantry 20.

The display controller 78 controls display of the operation panel 39. The display controller 78 displays guide information on the operation panel 39 based on information acquired via the communication module 74.

### Example 2 of Additional Configuration

In a case where the bed 30B is configured to comprise the electrically assisted caster, the processor 70 may perform control of turning off an electrical assistance function or reducing an output of the electrically assisted caster once the bed 30B approaches the docking portion 24. Accordingly, collision of the bed 30B with the docking portion 24 can be suppressed with a large momentum.

### Advantages of Second Embodiment

According to the second embodiment, a load on the operator of the bed 30B can be reduced as in the first embodiment. In addition, according to the second embodiment, since guide information is displayed on the operation panel 39 at the hand of the operator who pushes the bed 30B, the operator easily visually recognizes the guide information.

### Third Embodiment

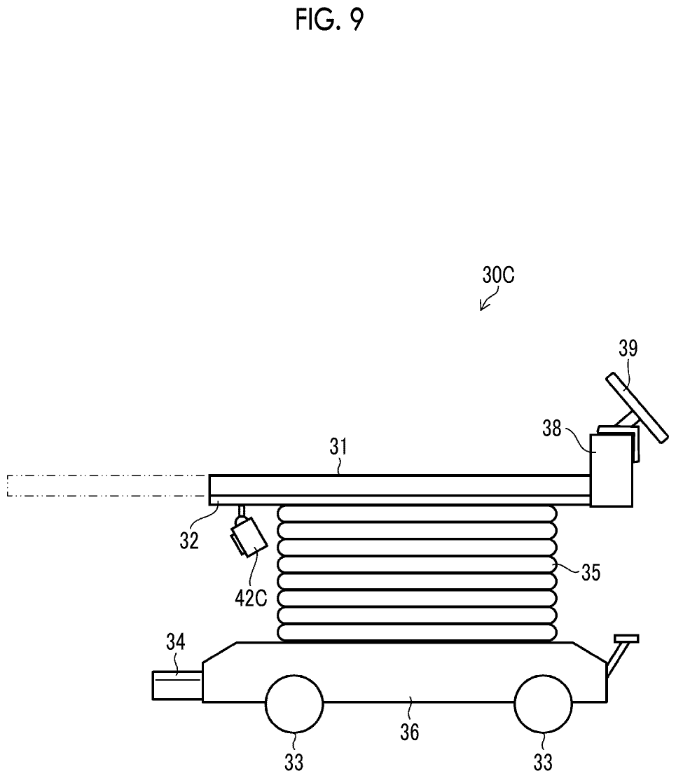

FIG. 9 is a schematic side view schematically showing a configuration of a bed 30C according to a third embodiment. Regarding the configuration shown in FIG. 9, elements common to those in FIGS. 2 and 7 are assigned with the same reference numerals, and redundant description will be omitted. In the third embodiment, an example in which the bed 30C comprises a camera 42C instead of the camera 42 described with reference to FIG. 1 will be described.

The camera 42C is disposed, for example, on a front side end portion of the bed 30C so that the docking portion 24 of the gantry 20A can be imaged. The number of cameras 42C disposed at the bed 30C is not limited to one, and there may be a plurality of cameras 42C. The gantry 20A to which the bed 30C is connected may be configured not to include the camera 42 (see FIG. 6) or may be configured not to comprise the communication module 66.

The bed 30C recognizes the docking portion 24 of the gantry 20A from an image captured by the camera 42C and displays guide information on the operation panel 39 based on a positional relationship ascertained by analyzing a position of the bed 30C with respect to the docking portion 24 through image analysis.

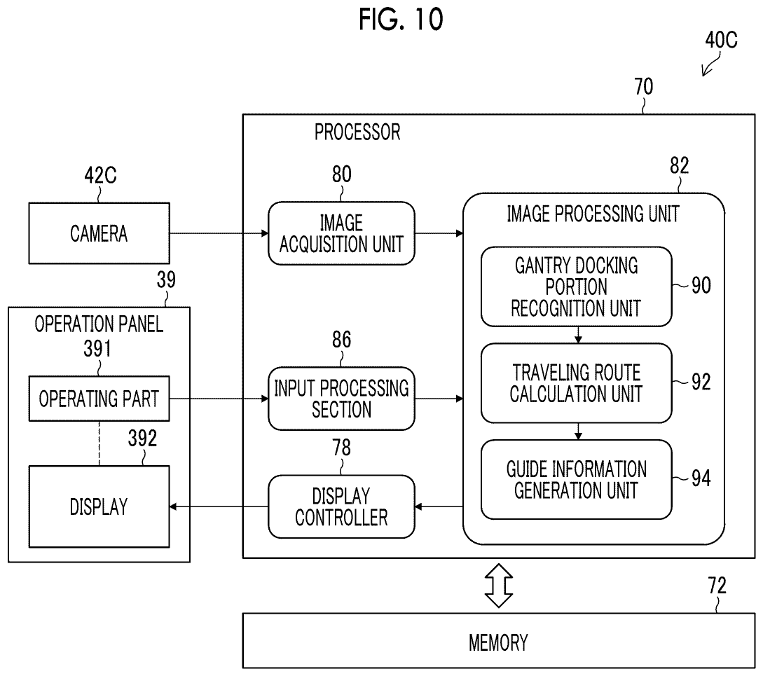

FIG. 10 is a functional block diagram of a bed induction system 40C incorporated in the bed 30C according to the third embodiment. In FIG. 10, elements having the configurations that are the same as or similar to configurations shown in FIG. 8 are assigned with the same reference numerals, and redundant description will be omitted.

The bed induction system 40C includes the camera 42C, the processor 70, the memory 72, and the operation panel 39. The operation panel 39 includes an operating part 391 and a display 392. The operating part 391 may be a touch panel integrated with the display 392. The operating part 391 may include an operation member such as a physical operation button, an input key, and a dial.

The processor 70 functions as an image acquisition unit 80, an image processing unit 82, an input processing section 86, and the display controller 78. The image acquisition unit 80 acquires an image captured by the camera 42C. The image processing unit 82 processes an image (camera image) obtained from the camera 42C. The image processing unit 82 includes a gantry docking portion recognition unit 90, a traveling route calculation unit 92, and a guide information generation unit 94.

The gantry docking portion recognition unit 90 recognizes the docking portion 24 of the gantry 20A from a camera image and ascertains a positional relationship between the docking portion 24 and the bed 30C. The gantry docking portion recognition unit 90 analyzes the camera image to ascertain a position and a posture of the bed 30C with respect to the docking portion 24.

The gantry docking portion recognition unit 90 may be configured to use, for example, a trained model trained to perform thing detection using a machine learning algorithm or may be configured to perform image recognition processing such as pattern matching.

Since the position of the camera 42C attached to the bed 30C is fixed and a positional relationship between the camera 42C and the docking portion 34 is also fixed, the gantry docking portion recognition unit 90 ascertains a positional relationship between the docking portion 24 of the gantry 20A and the docking portion 34 of the bed 30C. Information, such as a relative position of the bed 30C with respect to the docking portion 24 of the gantry 20A, and the posture and the traveling direction of the bed 30C, is acquired by the gantry docking portion recognition unit 90.

The traveling route calculation unit 92 and the guide information generation unit 94 may have a configuration of performing the same processing as the traveling route calculation unit 62 and the guide information generation unit 64 described with reference to FIG. 2. That is, the traveling route calculation unit 92 calculates a desired traveling route of the bed 30C in order to connect the docking portion 34 of the bed 30C to the docking portion 24 of the gantry 20A from a positional relationship between the gantry 20A and the bed 30C ascertained by the gantry docking portion recognition unit 90.

The guide information generation unit 94 generates guide information for inducing the bed 30C to the docking portion 24 of the gantry 20A based on calculation results of the traveling route calculation unit 92.

The input processing section 86 receives a signal input via the operating part 391 and performs processing corresponding to the received signal. For example, the input processing section 86 may receive a designation of activation or deactivation of the guide display function from the operating part 391 and control processing of the image processing unit 82 in accordance with the designated instruction.

The display controller 78 generates a display signal necessary for display output to the display 392 and controls display of the display 392. The display controller 78 displays guide information on the operation panel 39 based on processing results of the image processing unit 82.

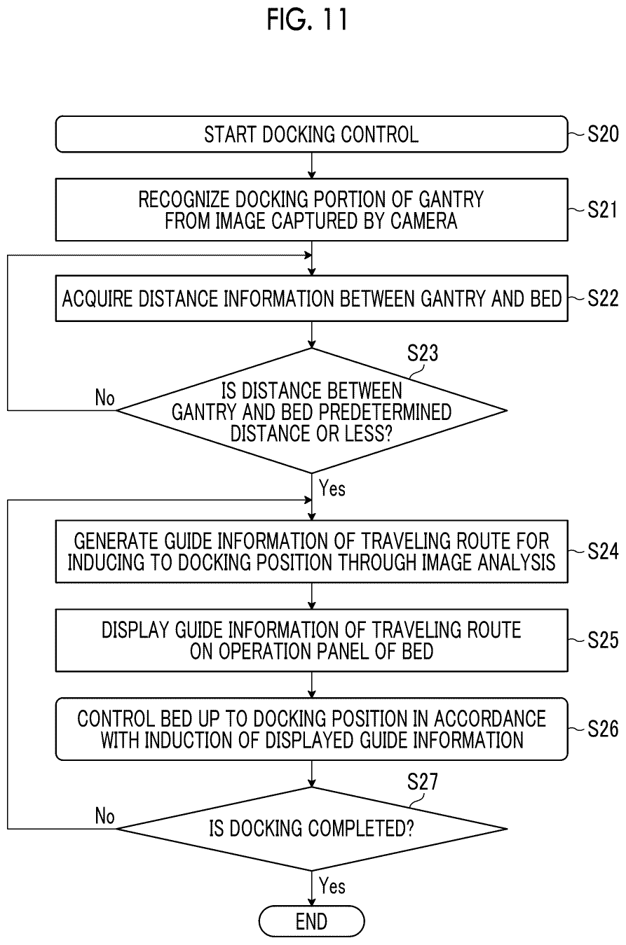

FIG. 11 is a flowchart showing an example of an operation of the bed induction system 40C according to the third embodiment. Step S20 is the same step as step S10 in FIG. 5.

In step S21, the processor 70 acquires an image captured by the camera 42C and recognizes the docking portion 24 of the gantry 20A from the camera image.

In step S22, the processor 70 ascertains a positional relationship between the gantry 20A and the bed 30C from the camera image and acquires distance information between the gantry 20A and the bed 30C.

Steps S23 and S24 are the same steps as steps S13 and S14 in FIG. 5. In step S23, the processor 70 determines whether or not a distance between the gantry 20A and the bed 30C has approached a predetermined distance or less. The predetermined distance may be a distance set as a guide display start determination distance described with reference to FIG. 3. Alternatively, the processor 70 may determine that the docking portion 24 has approached by the predetermined distance or less once the docking portion 24 is recognized from the camera image. In a case where the determination result in step S23 is No, the processor 70 proceeds to step S22.

In a case where the determination result in step S23 is Yes, the processor 70 proceeds to step S24.

In step S24, the processor 70 generates guide information indicating a traveling route for inducing the bed 30C to the docking position through image analysis on the camera image.

In step S25, the processor 70 displays the generated guide information on the operation panel 39 of the bed 30C. Accordingly, the operator of the bed 30C can control the bed 30C to the docking position in accordance with the induction of the guide information displayed on the operation panel 39.

Step S26 is the same step as step S16 in FIG. 5.

In step S27, the processor 70 determines whether or not the docking is completed. In a case where the determination result in step S27 is No (docking is not completed), the processor 70 returns to step S24.

In a case where the determination result in step S27 is Yes (docking completion), the processor 70 ends the guide display. The processor 70 may perform notification of notifying the operator that the docking is completed after the docking is completed.

### Advantages of Third Embodiment

According to the third embodiment, as in the second embodiment, a load on the operator of the bed 30C can be reduced, and the operator can easily visually recognize guide information through guide display on the operation panel 39. Further, according to the third embodiment, since the guide display is realized by the bed induction system 40C mounted on the bed 30C, it is not necessary to make any particular change or the like in a device configuration on a gantry 20A side.

In addition, according to the third embodiment, as the bed 30C comprises the camera 42C, there is an advantage in that an obstacle can be detected during movement in a place other than the examination room.

### Structural Example of Bed Comprising Electrically Assisted Caster and Steering Caster

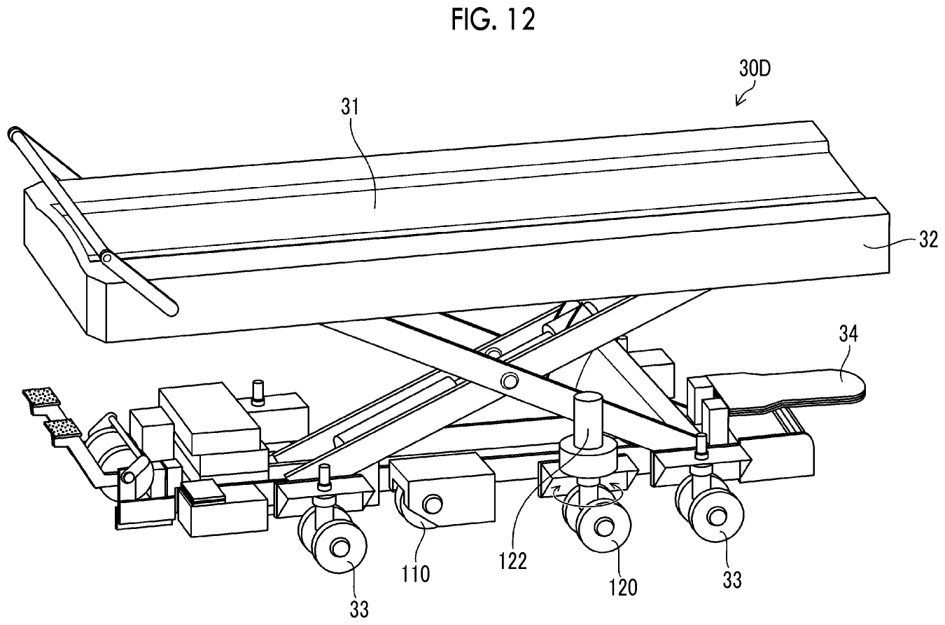

FIG. 12 is a perspective view showing a structural example of a bed 30D comprising an electrically assisted caster 110 and a steering caster 120. FIG. 12 shows a mechanical structure inside the bed covered with the bellows portion 35 and the cover 36 described with reference to FIG. 1.

The bed 30D shown in FIG. 12 comprises the electrically assisted caster 110 and the steering caster 120 in addition to the configuration described with reference to FIG. 1. The electrically assisted caster 110 is an electric wheel that includes a motor (not shown) which applies motive power for moving the bed 30D and that provides traveling assistance to the bed 30D. The electrically assisted caster 110 assists bed traveling by providing assistance to a person who pushes the bed 30D and may be configured not to completely self-travel with motive power of the electrically assisted caster 110.

The steering caster 120 is a wheel (caster) used in order to change a traveling direction of the bed 30D, and a motor 122 can control a swiveling orientation of the wheel. The steering caster 120 is controlled such that the bed 30D is directed to the docking position based on calculation results of the traveling route calculation unit 92.

In FIG. 12, an example of the bed 30D comprising the electrically assisted caster 110 and the steering caster 120 is shown, but a form in which one configuration of the electrically assisted caster 110 or the steering caster 120 is omitted is also possible. For example, a form of comprising the electrically assisted caster 110 without the steering caster 120 is also possible.

A configuration of comprising at least one configuration of the electrically assisted caster 110 or the steering caster 120 may be applied to any one of the bed 30 in the first embodiment, the bed 30B in the second embodiment, and the bed 30C in the third embodiment which are described above.

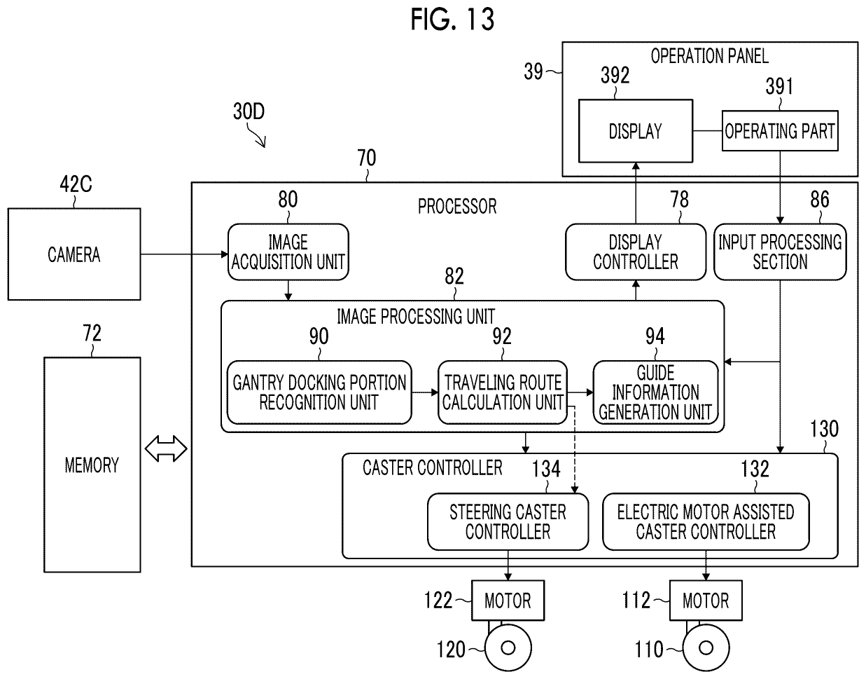

FIG. 13 is a functional block diagram of the bed 30D on which the same system as the bed induction system 40C according to the third embodiment is mounted.

In FIG. 13, elements common to the configurations described with reference to FIG. 10 are assigned with the same reference numerals, and redundant description will be omitted. The processor 70 functions as a caster controller 130. The caster controller 130 includes an electrically assisted caster controller 132 and a steering caster controller 134. The electrically assisted caster controller 132 controls a motor 112 of the electrically assisted caster 110. The steering caster controller 134 controls the motor 122 of the steering caster 120. Other configurations may be the same as the configurations described with reference to FIG. 10.

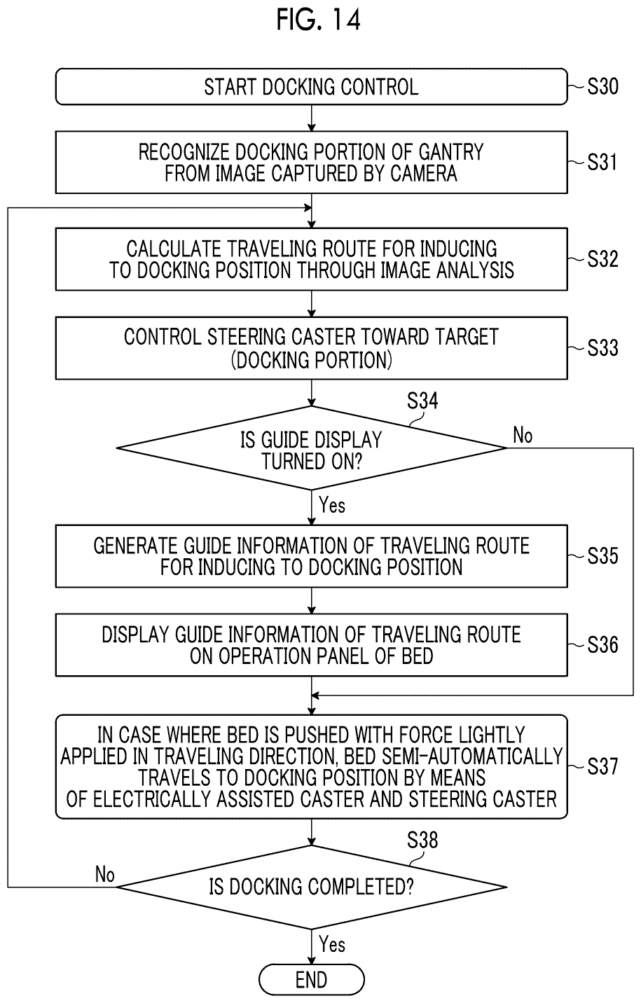

FIG. 14 is a flowchart showing example 1 of an operation of the bed 30D comprising the electrically assisted caster 110 and the steering caster 120. Steps S30 and S31 are the same steps as steps S20 and S21 in FIG. 11. By starting docking control in step S30, the bed 30D travels with a manual force and motive power of the electrically assisted caster 110.

In step S32, the processor 70 calculates a traveling route for inducing the bed 30D to the docking position through image analysis on a camera image.

In step S33, the processor 70 controls the steering caster 120 such that the bed 30D is directed to the docking portion 24, which is the target, based on the calculation result of the traveling route.

In step S34, the processor 70 determines whether or not to perform guide display on the operation panel 39. This determination may be to determine whether or not a distance between the bed 30D and the gantry 20A is a predetermined distance or less, as in step S23 in FIG. 11, or may be to determine whether or not the guide display function is activated/deactivated.

In a case where the determination result in step S34 is No, the processor 70 proceeds to step S37.

In step S37, in a case where the operator lightly applies a force to push the bed 30D in the traveling direction, the bed 30D semi-automatically travels to the docking position by means of the electrically assisted caster 110 and the steering caster 120.

In a case where the determination result in step S34 is Yes, the processor 70 proceeds to step S35. In step S35, the processor 70 generates guide information indicating a traveling route for inducing the bed 30D to the docking position based on the calculation result in step S32.

In step S36, the processor 70 displays the generated guide information on the operation panel 39. Accordingly, in step S37, the operator of the bed 30D can control the bed 30D to the docking position in accordance with the induction of the guide information displayed on the operation panel 39.

In step S38, the processor 70 determines whether or not the docking is completed. In a case where the determination result in step S38 is No (docking is not completed), the processor 70 returns to step S32.

In a case where the determination result in step S38 is Yes (docking completion), the processor 70 ends the guide display and the driving of the electrically assisted caster 110.

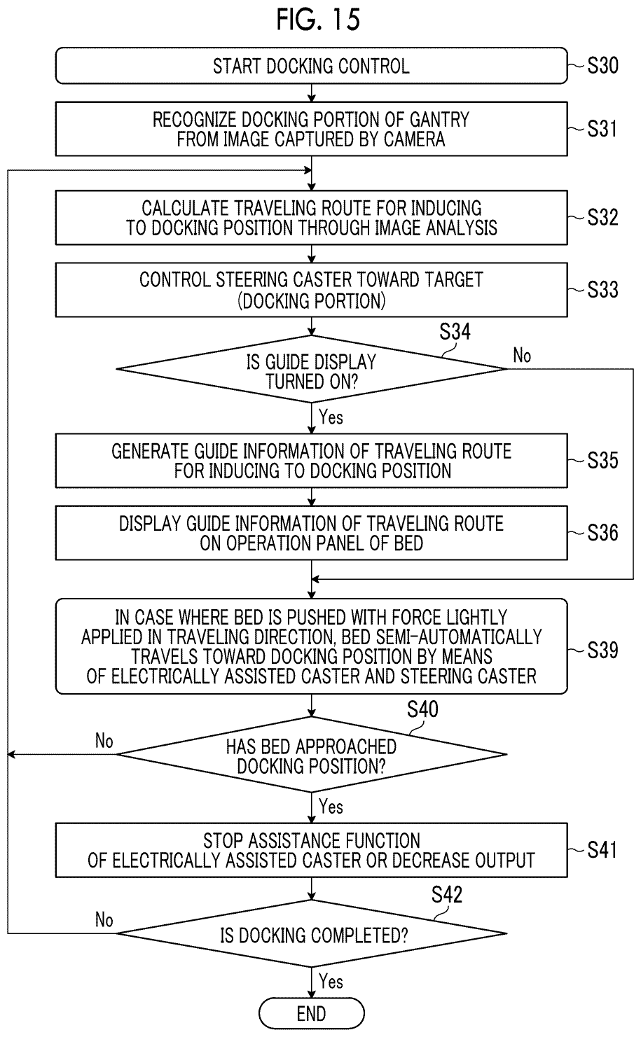

FIG. 15 is a flowchart showing example 2 of an operation of the bed 30D comprising the electrically assisted caster 110 and the steering caster 120.

Differences from FIG. 14 will be described with reference to FIG. 15. The flowchart of FIG. 15 includes steps S39, S40, S41, and S42 instead of steps S37 and S38 in FIG. 14.

In step S39, in a case where the operator lightly applies a force to push the bed 30D in the traveling direction, the bed 30D semi-automatically travels toward the docking position by means of the electrically assisted caster 110 and the steering caster 120.

In step S40, the processor 70 determines whether or not the bed 30D has approached the docking position by a predetermined distance or less. The predetermined distance herein may be set to a value different from the first distance set as the guide display start determination distance described with reference to FIG. 3 or may be set to the same value. For example, a second distance that is a determination criterion in step S40 may be set to a value smaller than the first distance.

In a case where the determination result in step S40 is No, the processor 70 proceeds to step S32. In a case where the determination result in step S40 is Yes, the processor 70 proceeds to step S41.

In step S41, the processor 70 performs control of stopping the assistance function of the electrically assisted caster 110 or decreasing an output. Accordingly, the assistance of the electrically assisted caster 110 is reduced before the bed 30D comes into contact with the docking portion 24, and an increase in impact in a case of docking is suppressed.

In step S42, the processor 70 determines whether or not the docking is completed. In a case where the determination result in step S42 is No (docking is not completed), the processor 70 returns to step S32.

In a case where the determination result in step S42 is Yes (docking completion), the processor 70 ends the guide display. In addition, in a case where the assistance function of the electrically assisted caster 110 is not stopped in step S41 (in a case where the output is decreased and the assistance function is activated), the processor 70 stops the electrically assisted caster 110 and ends the flowchart of FIG. 15.

### Regarding Hardware Configuration of Each Processing Section

Hardware structures of processing sections (processing units) that execute various types of processing, such as the image acquisition unit 50, the image processing unit 52, the bed recognition unit 60, the traveling route calculation unit 62, the guide information generation unit 64, and the display controller 54, which are shown in FIG. 2, the communication controller 68, the communication controller 76, and the display controller 78, which are shown in FIG. 8, the image acquisition unit 80, the image processing unit 82, the input processing section 86, the gantry docking portion recognition unit 90, the traveling route calculation unit 92, the guide information generation unit 94, and the display controller 54, which are shown in FIG. 10, the caster controller 130, the electrically assisted caster controller 132, and the steering caster controller 134, which are shown in FIG. 13, are, for example, various types of processors as described below.

Various types of processors include a CPU that is a general-purpose processor which executes a program and which functions as various types of processing sections, a GPU, a programmable logic device (PLD) that is a processor whose circuit configuration is changeable after manufacturing, such as a field programmable gate array (FPGA), and a dedicated electric circuit that is a processor having a circuit configuration exclusively designed to execute specific processing, such as an application specific integrated circuit (ASIC).

One processing section may be composed of one of the various types of processors or may be composed of two or more processors of the same type or different types. For example, one processing section may be composed of a plurality of FPGAs, a combination of a CPU and an FPGA, or a combination of a CPU and a GPU. In addition, one processor may constitute a plurality of processing sections. Examples of the plurality of processing sections composed of one processor include, first, as represented by a computer such as a client and a server, a form in which one processor is composed of a combination of one or more CPUs and software and the processor functions as the plurality of processing sections. Second, there is a form in which, as represented by a system on chip (SoC) and the like, a processor that realizes functions of the entire system including a plurality of processing sections with one integrated circuit (IC) chip is used. As described above, the various types of processing sections are composed of one or more of the various types of processors used as a hardware structure.

Further, as the hardware structure of the various types of processors, more specifically, an electric circuit (circuitry) in which circuit elements such as semiconductor elements are combined is used.

Regarding Program that Operates Computer

A program causing a computer to realize a part or all of the processing functions in the above processors 46 and 70 according to each embodiment described above can be recorded on a computer-readable medium which is an optical disk, a magnetic disk, or a tangible non-transitory information storage medium other than a semiconductor memory, and the program can be provided through this information storage medium.

In addition, instead of an aspect in which the program is stored and provided in such a tangible non-transitory computer-readable medium, a program signal can be provided as a download service by using an electric telecommunication line, such as the Internet.

Further, a part or all of the processing functions of the processors 46 and 70 may be realized by cloud computing or may also be provided as software as a service (SaaS).

### Other Application Examples

A technique according to the embodiment of the present disclosure can be applied to a medical apparatus to which a dockable bed is connected and/or a bed regardless of the type of medical apparatus. The medical apparatus is not limited to an apparatus that captures a medical image, and may be a determination device that does not have an imaging function, or may be a therapeutic device, such as a surgical support robot.

### Modification Example of Display Device

The form of the display device that displays guide information is not limited to the examples of the gantry monitor (display device 44) and the operation panel 39 described above, and various forms are possible. For example, the display device that displays guide information may be a stationary display disposed in a room where the medical apparatus is placed or may be a wearable display worn by the operator of the bed. In addition, the display device may be a projector. A plurality of display devices of the same type or different types may be used in combination.

### Others

The technical scope of the present invention is not limited to the scope described in the embodiments. The configuration in each embodiment and the configuration and the like in the modification example can be combined between the respective embodiments as appropriate without departing from the gist of the present invention.

### EXPLANATION OF REFERENCES

10, 10A, 10B: medical image diagnosis system
20, 20A: gantry
21: gantry cover
22: imaging space
24: docking portion
30, 30B, 30C, 30D: bed
31: top plate
32: top plate holding part
33: wheel
34: docking portion
35: bellows portion
36: cover
38: handle portion
39: operation panel
40, 40C: bed induction system
42, 42A, 42C: camera
44: display device
46: processor
48: memory
50: image acquisition unit
52: image processing unit
54: display controller
60: bed recognition unit
62: traveling route calculation unit
64: guide information generation unit
66, 74: communication module
68, 76: communication controller
70: processor
72: memory
78: display controller
80: image acquisition unit
82: image processing unit
86: input processing section
90: gantry docking portion recognition unit
92: traveling route calculation unit
94: guide information generation unit
110: electrically assisted caster
112, 122: motor
120: steering caster
130: caster controller
132: electrically assisted caster controller
134: steering caster controller
391: operating part
392: display
AR1, AR2: arrow
LN: line
S10 to S17: steps of operation of bed induction system according to first embodiment
S20 to S27: steps of operation of bed induction system according to third embodiment
S30 to S42: steps of operation of bed comprising electrically assisted caster and steering caster

What is claimed is:

1. A bed induction system comprising:
one or more sensors that obtain information on a positional relationship between a medical apparatus having a docking portion to which a bed is connected and the bed;
one or more processors that execute processing of displaying guide information for inducing the bed to the docking portion based on the information obtained from the one or more sensors; and
one or more display devices that display the guide information.

2. The bed induction system according to claim 1,
wherein the medical apparatus is a gantry of a medical imaging apparatus that captures a medical image of a subject.

3. The bed induction system according to claim 1,
wherein the one or more sensors are disposed at at least one of the medical apparatus or the bed.

4. The bed induction system according to claim 1,
wherein the one or more sensors are disposed at at least one of a ceiling or a wall of a room where the medical apparatus is disposed.

5. The bed induction system according to claim 1,
wherein the one or more sensors include a camera.

6. The bed induction system according to claim 5,
wherein the one or more processors ascertain the positional relationship between the medical apparatus and the bed by analyzing an image obtained from the camera.

7. The bed induction system according to claim 1,
wherein the one or more display devices are disposed at at least one of the medical apparatus or the bed.

8. The bed induction system according to claim 1,
wherein the one or more processors are configured to:
generate the guide information based on the information obtained from the one or more sensors.

9. The bed induction system according to claim 1,
wherein the guide information includes information indicating a traveling route for guiding the bed to the docking portion.

10. The bed induction system according to claim 1,
wherein the guide information includes information indicating a dockable angle range of the bed with respect to the docking portion.

11. The bed induction system according to claim 1,
wherein the information on the positional relationship includes distance information between the medical apparatus and the bed.

12. The bed induction system according to claim 1,
wherein the one or more processors are configured to:
display the guide information on the one or more display devices in a case where a distance between the medical apparatus and the bed is a first distance or less.

13. The bed induction system according to claim 12,
wherein the one or more processors are configured to:
in a case where display of the guide information on the one or more display devices is started, maintain the display of the guide information until docking completion.

14. The bed induction system according to claim 1,
wherein the one or more processors are configured to:
hide the guide information in a case of undocking, which is separating the bed from the medical apparatus, and activate a guide display function after a predetermined time elapses from the undocking.

15. The bed induction system according to claim 1,
wherein the bed includes an electrically assisted caster that provides traveling assistance to the bed, and
the one or more processors are configured to:
perform control of reducing the traveling assistance of the electrically assisted caster in a case where a distance between the medical apparatus and the bed is a second distance or less.

16. The bed induction system according to claim 1,
wherein the bed includes a steering caster that changes a traveling direction of the bed, and
the one or more processors are configured to:
control the steering caster so that the bed is directed to the docking portion based on the information obtained from the one or more sensors.

17. A medical apparatus comprising:
a docking portion to which a bed is connected;
one or more sensors that obtain information on a positional relationship with the bed;
one or more processors that execute processing of displaying guide information for inducing the bed to the docking portion based on the information obtained from the one or more sensors; and
one or more display devices that display the guide information.

18. A bed that is attachable to and detachable from a docking portion provided at a medical apparatus, the bed comprising:
one or more sensors that obtain information on a positional relationship between the medical apparatus and the bed;
one or more processors that execute processing of displaying guide information for inducing the bed to the docking portion based on the information obtained from the one or more sensors; and
one or more display devices that display the guide information.

19. A medical image diagnosis system comprising:
a gantry that has a docking portion to which a bed is connected;
the bed that is attachable to and detachable from the docking portion;
one or more sensors that obtain information on a positional relationship between the gantry and the bed;
one or more processors that execute processing of displaying guide information for inducing the bed to the docking portion based on the information obtained from the one or more sensors; and
one or more display devices that display the guide information.
